(19) United States
(12) Patent Application Publication
(10) Pub. No.: US 2013/0177606 A1
(43) Pub. Date: Jul. 11, 2013

(54) TRANSDERMAL THERAPEUTIC SYSTEM WITH CHOLINESTERASE INHIBITOR

(71) Applicants: ALFRED E. TIEFENBACHER (GmbH & Co. KG), Hamburg (DE); AMW GmbH, Wamgau (DE)

(72) Inventors: Tobias PFALLER, Holzkirchen (DE); Susanne LANG, Holzkirchen (DE); Florian SAHR, Holzkirchen (DE); Katharina WOLFF, Hamburg (DE); Ansgar FITZNER, Hamburg (DE)

(73) Assignees: AMW GMBH, Warngau (DE); ALFRED E. TIEFENBACHER (GMBH & CO. KG), Hamburg (DE)

(21) Appl. No.: 13/738,430

(22) Filed: Jan. 10, 2013

(30) Foreign Application Priority Data
Jan. 11, 2012 (DE) 10 2012 000 369.2

(51) Int. Cl. A61K 9/70; A61K 31/27

(52) U.S. Cl. CPC A61K 9/7084; A61K 31/27; A61K 9/7092
USPC 424/400; 514/490

(57) ABSTRACT

The invention concerns a transdermal therapeutic system with a carbamate cholinesterase inhibitor for the treatment of mild to moderate Alzheimer's disease dementia and/or Parkinson's disease dementia.

### FIELD OF THE INVENTION

This invention concerns a transdermal therapeutic system (TDS) which contains a cholinesterase inhibitor of the carbamate type as an active ingredient, for example rivastigmine.

### BACKGROUND OF THE INVENTION

To administer rivastigmine via a transdermal system (TDS), according to the prior art an antioxidant is required, since without it, the active ingredient does not remain chemically stable. EP 1 047 409 B1, WO 99/34 782 and the Exelon® Patch (rivastigmine transdermal system) teach using alpha-tocopherol, for example, as an antioxidant stabilizer. However, the literature further describes that the antioxidant alpha-tocopherol can result in allergic contact dermatitis. Examples of references made to this are in Dermatitis Jun. 21, 2010 (3): 148-53 (Kosari, P., Alikhan, A., Sockolov, M. & Feldman, S R.: Vitamin E and allergic contact dermatitis), Dermatitis Aug. 21, 2010 (4): 199-202 (Adams, A K. & Connolly, S M.: Allergic contact dermatitis from vitamin E: the experience at Mayo Clinic Arizona 1987 to 2007) and Dermatitis May-June 19, 2008 (3): 154-6 (Ramirez Santos, A., Fernández-Redondo, V., Pérez Pérez, L., Concheiro Cao, J. & Toribio, J: Contact allergy from vitamins in cosmetic products).

In addition, with the transdermal therapeutic systems according to the prior art, a silicone adhesive layer is required to achieve a strong enough adhesion to the skin. For example, in WO 2007/064 407 A1, it is described that the reservoir layer (such as with Exelon) has too little adhesive power and for that reason, lamination of a silicon adhesive layer was required.

DE 38 05 744 C2 and DE 38 44 992 B4 describe the use of the S-(-)-enantiomer of rivastigmine and a salt thereof for systematic transdermal administration for the treatment of dementia and Alzheimer's disease.

EP 1 171 104 B1 and WO 00/64 418 describe a transdermal therapeutic matrix or a reservoir system with a basic or neutral active ingredient, e.g. rivastigmine, as well as a completely or partially neutralized acidic pressure sensitive polyacrylate adhesive agent

EP 2 292 219 A1, EP 1 959 937 and EP 2 286 802 describe a transdermal therapeutic system with rivastigmine with an AUC₂₄ₕ from 25 to 450 ng×h/mL after repeated once daily dosage.

WO 2011/076 621 A2 further describes a transdermal therapeutic system with rivastigmine as the active ingredient in a reservoir with a polymer matrix, which features neither hydroxyl nor carboxyl groups.

DE 698 30 095 T2 describes a cosmetic or dermopharmaceutical plaster for release of a water soluble active ingredient present in the form of particles and additionally, a fat soluble active ingredient dissolved in an oil, into the epidermis [0001]. Avocado oil was proposed among others in a large list of oils. Avocado oil is also proposed in DE 198 82 916 T1 (page 7), in the same way in a list of oils for skin care and for skin protection, although here in a gel composition. Avocado oil can also be employed in the mechanism described in DE 199 11 262 A1 for the release of cosmetic active ingredients, where it is again quoted in a long list of oils. Avocado oil is finally mentioned in DE 600 28 642 T2 in a comprehensive list of oils for incorporation of a solubilized active ingredient [0020], and namely for a plaster containing magnetic particles, which promote the penetration of the active substance into the skin [0009], where a cosmetic effect and/or a pharmaceutical treatment with the plaster on the skin is to be conducted [0001].

The use of avocado oil was with a transdermal system was mentioned several years ago, but has not been developed since. For example, DE 33 47 278 A1 (filing date 28.12.1983) and DE 34 09 079 A1 (filing date 13.03.1984) describe an anti-inflammatory medicinal plaster, for which avocado oil, among others, was proposed as an entrainer in a list of oils (page 12 or page 9). DE 42 37 453 C1 (filing date 06.11.1992) further describes a transdermal system for the release of a special active ingredient, namely 17-β-estradiol, whereby avocado oil is proposed in an extensive list of additives that could possibly improve solubility or for improvement of skin permeation (page 2 line 64 to page 3 line 13).

### SUMMARY OF THE INVENTION

The present invention is drawn to a transdermal therapeutic system comprising
an active ingredient reservoir layer which contains
   at least one carbamate cholinesterase inhibitor as the active ingredient, at least one polyacrylate polymer with free carboxyl groups and at least one polyacrylate polymer with quaternary ammonia groups, and
a silicone-free adhesive layer comprising at least one polyacrylate with free hydroxyl groups;
wherein said active ingredient reservoir layer does not contain an antioxidant.

The present invention is further drawn to methods of treatment with the transdermal system.

### DETAILED DESCRIPTION OF THE INVENTION

The present invention is drawn to a transdermal therapeutic system comprising
an active ingredient reservoir layer which contains
   at least one carbamate cholinesterase inhibitor as the active ingredient, at least one polyacrylate polymer with free carboxyl groups and at least one polyacrylate polymer with quaternary ammonia groups, and
a silicone-free adhesive layer comprising at least one polyacrylate with free hydroxyl groups;
wherein said active ingredient reservoir layer does not contain an antioxidant.

The present invention is further drawn to methods of treatment with the transdermal system.

In particular, the present invention provides a transdermal therapeutic system for the treatment of mild to moderate Alzheimer's disease-type dementia and/or Parkinson's disease-type dementia, and to provide a treatment of mild to moderate Alzheimer's disease-type dementia and/or Parkinson's disease-type dementia, which contains a carbamate cholinesterase inhibitor, such as rivastigmine.

The transdermal therapeutic system of the invention provides the features of: (1) avoidance of frequent to very frequent side effects associated with oral administration, particularly nausea, diarrhoea and vomiting. (2) The transdermal therapeutic system should also exhibit a better compliance than oral medications. (3) Thus the transdermal therapeutic system should allow for convenient application on the skin and (4) offer good skin compatibility with good wearing comfort and superior adhesive power. Furthermore, it should offer (5) a high storage stability and (6) reliable efficacy. (7) The transdermal therapeutic system should be able to be manufactured cost-effectively. (8) It should be especially be applicable for 1 to 7 days for a long, sustained, continuous release of the active ingredient. (9) Finally, the transdermal therapeutic system should also be able to be provided without antioxidant.

The present invention is thus drawn to a transdermal therapeutic system comprising
an active ingredient reservoir layer which contains
   at least one carbamate cholinesterase inhibitor as the active ingredient, at least one polyacrylate polymer with free carboxyl groups and at least one polyacrylate polymer with quaternary ammonia groups,
a silicone-free adhesive layer comprising at least one polyacrylate with free hydroxyl groups;
wherein said active ingredient reservoir layer does not contain an antioxidant.

According to the invention, rivastigmine can be the carbamate cholinesterase inhibitor for the transdermal therapeutic system. The active ingredient can also be a physiologically compatible salt, hydrate, solvate or derivate thereof.

In addition, according to the invention rivastigmine and/or at least one other carbamate cholinesterase inhibitor can be used as the active ingredient for the transdermal therapeutic system.

According to the invention, an active ingredient concentration of 0.1% to 40% and particularly up to 50% by weight (based on the total weight of the active ingredient reservoir) can be envisaged for the transdermal therapeutic system.

In addition, according to the invention, an active ingredient concentration of 15% to 35% and especially about 30% by weight (based on the total weight of the active ingredient reservoir) can be envisaged for the transdermal therapeutic system.

A minimum of one carbamate cholinesterase inhibitor can be present in the active ingredient reservoir in a dissolved or homogenously dispersed state in the inventive transdermal therapeutic system.

In addition, the transdermal therapeutic system may contain in the active ingredient reservoir layer 0% to 15% avocado oil and/or palm oil by weight (based on the total weight of the active ingredient reservoir). It is further contemplated that the transdermal therapeutic system can contain avocado oil and/or palm oil with additional content of at least one further facultative excipient, which contributes to improved cohesion or to improved solubility, for example, and particularly to the improved solubility of the active ingredient.

The avocado oil and/or palm oil and/or the facultative additional excipient can be present in the active ingredient reservoir in a dissolved or homogenously dispersed state in the inventive therapeutic system.

With the transdermal therapeutic system of the invention, the active ingredient reservoir layer and/or the adhesive layer contain one or more facultative pressure sensitive acrylate polymers. Various suitable acrylate polymers are commercially available and include, DuraTak 87-2353, which is disclosed in U.S. 2010/0 087 768 A1 page 5 [0085], as being a “random copolymer of 2-ethylhexylacrylate (2-EHA) 62.2%, methylacrylate (MA) 32.0%, glacial acrylic acid (GAA) 5.7% and glycidylmethacrylate (GMA) 0.03% in an organic solvent solution consisting of ethylacetate and hexane in the ratio 86.9:13.1, the copolymer is supplied at 35-38% solid in solution.” In addition, the following acrylate polymers are commercially available and described as follows.

| DuroTak | 387-2051 | 87-2353 |
|---|---|---|
| WO 99/34 782 A1 | Page 2 Para 3 with | Page 2 Para 3 |
| | Page 7 Para 3 | |
| WO 00/644 418 A2 | Page 15 Para 2 | |
| US 2010/0 087 768 A1 | Page 4 Table 1 | Page 5 [0085] |
| WO 2007/064 407 A1 | | Page 6 Para 4, |
| | | Page 12 Example |

In the inventive transdermal therapeutic system, the active ingredient reservoir can contain one or more facultative pressure sensitive polymers, particularly one or more polyacrylate polymers, preferably polyacrylates with free carboxyl groups, particularly DuroTak 87-235A or 235A (acrylic-based non-curing pressure sensitive adhesives with free carboxyl groups), and/or polyacrylate with free hydroxyl groups, particularly DuroTak 387-2510, 87-2510 or 2510 (acrylic-cased non-curing pressure sensitive adhesives with free hydroxyl groups), and/or polyacrylates with quaternary ammonium groups, or a facultative pressure sensitive mixture of several of these polymers.

The polyacrylates with quaternary ammonium groups suitable for the inventive transdermal therapeutic system can contain one or more copolymer of ethylacrylate, methyl methacrylate and methacryl acid ester with quaternary ammonium groups, preferably trimethylammonioethyl-methacrylate-chloride. Examples of the polyacrylate with quaternary ammonium groups include, for example, Eudragit RL 100, Eudragit RS 100, Eudragit RL PO and/or Eudragit RS PO. EUDRAGIT®RL 100/RL PO and EUDRAGI® RS 100/RS PO are copolymers of ethyl acrylate, methyl methacrylate and a low content of a methacrylic acid ester with quaternary ammonium groups (trimethylammonioethyl methacrylate chloride). The ammonium groups are present as salts and make the polymers permeable. The molar ratio of ethyl acrylate, methyl methacrylate and trimethylammonioethyl methacrylate is approx. 1:2:0.2 in EUDRAGIT RL and approx. 1:2:0.1 in EUDRAGIT RS.

The following reference further disclosure the general use of Eudragit polyacrylate polymers:
DE 39 39 376, DE 40 20 144 and DE 43 10 012;
EP 0 617 972, EP 0 848 960 and EP 1 061 900;
WO 99/34 782 A1 page 3 and WO 06/12 966.

For the inventive transdermal therapeutic system, the active ingredient reservoir layer can contain one or more of the following
polyacrylate with free carboxyl groups
polyacrylate with free hydroxyl groups
polyacrylate with quaternary ammonium groups
   which can be present in with the respective ratios of (3 to 4) : (4 to 1) : (0 to 2) and add up to 100% (based on weight).

Furthermore, the polyacrylate or polyacrylate mixture of the active ingredient reservoir of the inventive transdermal therapeutic system can be modified with a tackifier based on natural or synthetic hydrocarbon.

Furthermore, the active ingredient reservoir layer of the inventive transdermal therapeutic system can be provided with an adhesive layer, which can also optionally contain a a carbamate cholinesterase inhibitor as an active ingredient, wherein the carbamate cholinesterase inhibitor may be the same as that contained in the active ingredient reservoir layer and is preferably at least rivastigmine.

The active ingredient concentration in the adhesive layer may be in an amount of about 0.1% to 40% and particularly up to 50% by weight, and preferably 15% to 35% and particularly about 30% by weight (based on the total weight of the active ingredient reservoir).

Thus, in one embodiment of the invention, the transdermal therapeutic system may contain,
an active ingredient reservoir layer having, in addition to the active ingredient of a carbamate cholinesterase inhibitor,
   one or more polyacrylate with free carboxyl groups, and
   one or more polyacrylate with quaternary ammonium groups,
   in respective ratios of (4 to 6) : (3 to 1) and adding up to 100% (based on weight); and
an adhesive layer having one or more
   polyacrylate with free hydroxyl groups in amount up to 100% (on the basis of weight), and
   optionally the active ingredient.

Notably, the active ingredient reservoir layer and facultative adhesive layer in the inventive transdermal therapeutic system, and particularly the active ingredient reservoir, can be free of antioxidants. Thus the inventive transdermal therapeutic system can be free of any antioxidant content, for example, when the active ingredient reservoir contains or is composed of an non-crosslinked polyacrylate that features a carboxyl group like DuroTak 87-235A.

Furthermore, the inventive transdermal therapeutic system can contain a solubilizer. Exemplified solubilizers include, but are not limited to, 1,2-propanediol, dimethylacetamide and/or N-methylpyrrolidone.

Furthermore, the inventive transdermal therapeutic system can contain a cohesion enhancer, including but not limited to polysiloxane.

Furthermore, the inventive transdermal therapeutic system can contain an adhesive force promoter, including but not limited to, one or more resins of natural or synthetic origin.

Furthermore, the inventive transdermal therapeutic system can contain a permeation enhancer.

Furthermore, the inventive transdermal therapeutic system can be adjusted to an active ingredient skin permeation in vitro of 10 μg (micrograms)/(h and system) to 5 mg/(h and system).

Furthermore, the active ingredient content of the inventive transdermal therapeutic system can be adjusted to a continuous active ingredient release for the duration of whole days in the range of 1 to 7 days.

Finally, the inventive transdermal therapeutic system can be provided with
an active ingredient reservoir surface weight of 30 to 150, preferably 35 to 60, more preferred 35 to 45 and most preferred about 40 g/m² and
a surface weight of 10 to 40 g/m² for the facultative adhesive layer free of the active ingredient on the side of application, or
a surface weight of 10 to 30 g/m² and particularly about 20 g/m² for the adhesive layer which contains a carbamate cholinesterase inhibitor as an active ingredient.

The detachable layer can be a protective layer which is impermeable to the active ingredient, for example, a siliconized polyester foil, which is removed before application to the skin.

Hereafter, the invention is further illustrated by the examples:

### EXEMPLIFIED EMBODIMENTS OF THE INVENTION

### Example 1

Rivastigmine was diluted in ethylacetate. A polyacrylate mixture was added to the resulting solution, and thereafter homogenized. The quantities (dry weight) are indicated in the following table. The mixture obtained was laid onto a polyester foil up to a weight of 60 g homogenizate/m². Thereafter the solvent was removed by drying the foil with the inlaid homogenizate was dried for 15 min at 60° C. A siliconized polyester foil was laminated onto the dried adhesive film.

| Component | [% weight] |
|---|---|
| Rivastigmine | 30 |
| Polyacrylate with free carboxyl groups | |
| DuroTak 235 | 30 |
| Polyacrylate with free hydroxyl groups | |
| DuroTak 2510 | 40 |
| Polyacrylate with quaternary ammonium groups | 0 |

### Example 2

Example 1 was repeated with the following formulation

| Component | [% weight] |
|---|---|
| Rivastigmine | 30 |
| Polyacrylate with free carboxyl groups | |
| DuroTak 235 | 40 |
| Polyacrylate with free hydroxyl groups | |
| Duro Tak 2510 | 10 |
| Polyacrylate with quaternary ammonium groups | |
| Eudragit RS | 20 |

### Example 3

Example 1 was repeated with the following formulation

| Component | [% weight] |
|---|---|
| Rivastigmine | 30 |
| Polyacrylate with free carboxyl groups | |
| DuroTak 235 | 40 |
| Polyacrylate with free hydroxyl groups | |
| DuroTak 2510 | 20 |
| Polyacrylate with quaternary ammonium groups | |
| Eudragit RS | 10 |

### Example 4

Example 1 was repeated with the following formulation for the active ingredient reservoir, whereby the polyacrylate mixture was laid on the polyester foil up to a weight of 40 g homogenizate/m².

| Component | [% weight] |
|---|---|
| Rivastigmine | 30 |
| Polyacrylate with free carboxyl groups | |
| DuroTak 235A | 50 |
| Polyacrylate with free hydroxyl groups | |
| DuroTak 2510 | 0 |
| Polyacrylate with quaternary ammonium groups | |
| Eudragit RL | 20 |

On the completed active ingredient reservoir an adhesive layer with the following composition was applied up to a weight of 20 g homogenizate/m²:

| Component | [% weight] |
|---|---|
| Rivastigmine | 30 |
| Polyacrylate with free carboxyl groups | 0 |
| Polyacrylate with free hydroxyl groups | |
| DuroTak 2510 | 70 |
| Polyacrylate with quaternary ammonium groups | |
| Eudragit | 0 |

### Example 5

Example 4 was repeated with the following formulation for the active ingredient reservoir, whereby the polyacrylate mixture was laid on the polyester foil up to a weight of 40 g homogenizate/m².

| Component | [% weight] |
|---|---|
| Rivastigmine | 30 |
| Polyacrylate with free carboxyl groups | |
| DuroTak 235A | 50 |
| Polyacrylate with free hydroxyl groups | |
| DuroTak 2510 | 0 |
| Polyacrylate with quaternary ammonium groups | |
| Eudragit RS | 20 |

An adhesive layer was applied to the completed active ingredient reservoir with the components according to example 4 up to a weight of 20 g homogenizate/m²:

1. Transdermal therapeutic system comprising
an active ingredient reservoir layer which contains
at least one carbamate cholinesterase inhibitor as the active ingredient, at least one polyacrylate polymer with free carboxyl groups and at least one polyacrylate polymer with quaternary ammonia groups,
a silicone-free adhesive layer comprising at least one polyacrylate with free hydroxyl groups;
wherein said active ingredient reservoir layer does not contain an antioxidant.

2. The transdermal therapeutic system according to claim 1 wherein said carbamate cholinesterase inhibitor is rivastigmine.

3. The transdermal therapeutic system according to claim 1 wherein said active ingredient in the active ingredient reservoir layer is present in an amount of about of 0.1% to 40% by weight (based on the total weight of the active ingredient reservoir).

4. The transdermal therapeutic system according to claim 1 wherein said adhesive layer further comprises the active ingredient.

5. The transdermal therapeutic system according to claim 1, wherein the at least one carbamate cholinesterase inhibitor has been dissolved or homogenously dispersed in the active ingredient reservoir layer.

6. The transdermal therapeutic system according to claim 1, wherein the active ingredient reservoir layer further comprises up to 15% weight avocado oil and/or palm oil (based on the total weight of the active ingredient reservoir).

7. The transdermal therapeutic system according to claim 1 further comprising at least one excipient in the active ingredient reservoir layer which improves cohesion and/or solubility of the active ingredient.

8. The transdermal therapeutic system according to claim 1, wherein the polyacrylate with quaternary ammonium groups is a copolymer containing ethylacrylate, methyl methacrylate and methacryl acid ester.

9. The transdermal therapeutic system according to claim 8, wherein the polyacrylate with quaternary ammonium groups in the active ingredient reservoir layer is trimethylammonioethyl-methylacrylate-chloride.

10. The transdermal therapeutic system according to claim 1, wherein the polyacrylate with free carboxyl groups in the active ingredient reservoir layer is DuroTak 87-235A.

11. The transdermal therapeutic system according to claim 1, wherein the polyacrylate with free hydroxyl groups in the adhesive layer is DuroTak 87-2510.

12. The transdermal therapeutic system according to claim 1, wherein the active ingredient reservoir layer optionally further comprises at least one polyacrylate with free hydroxyl groups.

13. The transdermal therapeutic system according to claim 12, wherein the polyacrylate with free carboxyl groups, polyacrylate with free hydroxyl groups and polyacrylate with quaternary ammonium groups are present in the active ingredient reservoir layer in the following respective ratio, 3 to 4 parts:4 to 1 parts: 0 to 2 parts, wherein the total is 100% (based on total weight of the polyacryates components).

14. The transdermal therapeutic system according to claim 1 wherein said polyacrylate with quaternary ammonium groups is at least one selected from the group consisting of Ammonio Methacrylate Copolymer Type A and/or Ammonio Methacrylate Copolymer Type B.

15. The transdermal therapeutic system according to claim 1 wherein the at least one polyacrylate polymer with free carboxy groups and at least one polyacrylate polymer with quaternary ammonia groups in the active ingredient reservoir layer is modified with a tackifier based on a natural or synthetic hydrocarbon.

16. The transdermal therapeutic system according to claim 1 wherein the system is adjusted to an active ingredient skin permeation in vitro of 10pg (microgram)/(h and system) to 5 mg/(h and system).

17. The transdermal therapeutic system according to claim 1, wherein the content of the active ingredient is adjusted for a continuous active ingredient release for 1 to 7 days.

18. Transdermal therapeutic system comprising
an active ingredient reservoir layer which contains
about 30% by weight of rivastigmine,
about 50% by weight of at least one polyacrylate polymer with free carboxyl groups, and
about 20% by weight of at least one polyacrylate polymer with quaternary ammonia groups,
a silicone-free adhesive layer comprising about 70% by weight of at least one polyacrylate with free hydroxyl groups;
wherein said reservoir layer does not contain an antioxidant.

19. Transdermal therapeutic system comprising
an active ingredient reservoir layer which contains
about 30% by weight of rivastigmine,
about 50% by weight of at least one polyacrylate polymer with free carboxyl groups, and
about 20% by weight of at least one polyacrylate polymer with quaternary ammonia groups,
a silicone-free adhesive layer comprising about 70% by weight of at least one polyacrylate with free hydroxyl groups and about 30% by weight of rivastigmine;
wherein said reservoir layer does not contain an antioxidant.

20. A method of treating dementia associated with Alzheimer's disease or Parkinson's disease, which comprises administering to a patient in need thereof the transdermal therapeutic system according to claim 1.
